(19) United States
(12) Patent Application Publication
(10) Pub. No.: US 2019/0231511 A1
(43) Pub. Date: Aug. 1, 2019

(54) WOVEN GRAFT COMPOSITE WITH VARIED DENSITY

(71) Applicant: THE SECANT GROUP, LLC, Telford, PA (US)

(72) Inventors: Seth A. WINNER, Durham, NC (US); Amanda WEBER, Macungie, PA (US); Andrew METZGER, Lafayette Hill, PA (US); Stephanie REED, Conshohocken, PA (US); Carissa SMOOT, Harleysville, PA (US)

(21) Appl. No.: 16/264,087

(22) Filed: Jan. 31, 2019

Related U.S. Application Data
(60) Provisional application No. 62/624,592, filed on Jan. 31, 2018.

(51) Int. Cl. A61F 2/07; A61F 2/00

(52) U.S. Cl. CPC A61F 2/07; A61F 2/0077; A61F 2250/0023; A61F 2240/001; A61F 2002/072

(57) ABSTRACT

The disclosure describes textiles for endovascular and other medical applications having a low-profile and varying density. The textiles are woven and are formed of a non-uniform construction. The resulting textiles have a low profile and areas of targeted density to enhance ingrowth of regenerative tissue.

### RELATED APPLICATIONS

This application claims the benefit of, and priority to, U.S. App. No. 62/624,592, filed Jan. 31, 2018, which is hereby incorporated by reference in its entirety.

### FIELD OF THE INVENTION

The present invention is directed to implantable thin-walled composite woven textiles having regions of variable density and/or porosity for cellular ingrowth.

### BACKGROUND OF THE INVENTION

In conventional woven implantable textiles, the textile is woven to have substantially uniform properties over the surface of the textile. Differing regions of the implanted textile encounter differing conditions based on their location.

### BRIEF DESCRIPTION OF THE INVENTION

It would be desirable for the properties of the implantable textile to vary throughout the textile in order to improve the medical outcomes of the patients receiving textile implants. Exemplary embodiments seek to overcome this and other limitations in textiles for medical and other applications by providing a lower profile textile having a variable porosity for use, for example, in woven endovascular grafts which exhibits the strength and permeability characteristics desirable for the intended application.

In an embodiment, a woven implantable textile having a first woven region having a first ends per inch in a warp and a first picks per inch in a weft defining a first density and a second woven region having a second ends per inch in the warp and a second picks per inch in the weft defining a second density. The woven construction of the first region and the woven construction of the second region are the same.

In an embodiment, a woven implantable textile having a first woven region formed from a first woven construction and a second woven region formed from a second woven construction. The woven construction of the first woven region and the woven construction of the second woven region are different.

Other features and advantages of the present invention will be apparent from the following more detailed description, taken in conjunction with the accompanying drawings which illustrate, by way of example, the principles of the invention.

### BRIEF DESCRIPTION OF THE DRAWINGS

FIG. 1 shows a textile having multiple regions of differing densities, in accordance with an exemplary embodiment.

FIG. 2 shows an uncoated textile, in accordance with an exemplary embodiment.

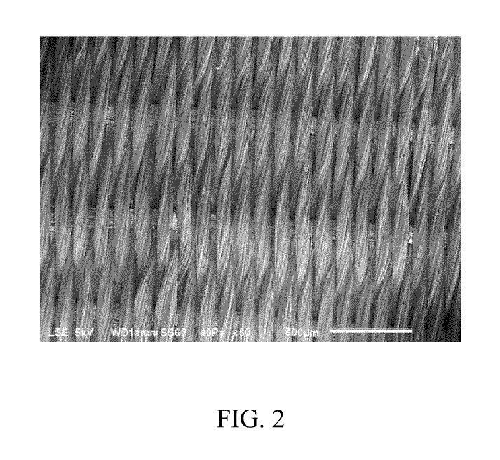

FIG. 3 shows a textile having a regenerative coating, in accordance with an exemplary embodiment.

FIG. 4 shows a textile exhibiting differing topography over two regions, in accordance with an exemplary embodiment.

FIG. 5 is a representation of a textile having regions of differing weaves and differing porosity, promoting differing rates of ingrowth, in accordance with an exemplary embodiment.

FIG. 6 shows a textile having both resorbable and non-resorbable weft yarns, in accordance with an exemplary embodiment.

FIG. 7 shows a coated deflected weft weave multi-dimensional textile, in accordance with an exemplary embodiment.

FIG. 8 shows an uncoated deflected weft weave multi-dimensional textile, in accordance with an exemplary embodiment.

FIG. 9 shows an uncoated textile having multiple weave types and including resorbable material in the weft, in accordance with an exemplary embodiment.

FIG. 10 is an illustration of the use of linear shaping, in accordance with an exemplary embodiment.

### DETAILED DESCRIPTION OF THE INVENTION

Provided is a vascular textile of varying porosity primarily for medical applications, including, for example, endovascular aneurysm repair (EVAR) and transcatheter valve replacement or repair (TVR) procedures.

Embodiments of the present disclosure, for example, in comparison to concepts failing to include one or more of the features disclosed herein, provide an implantable textile having a varying porosity throughout some or all of the textile.

During endovascular procedures, such as endovascular aneurysm repair and transcatheter aortic valve replacement procedures, an implantable textile may be inserted into the patient to reinforce the affected region of the blood vessel. Medical outcomes are affected by the rate of regenerative ingrowth of tissue into implanted textile. Variable density woven implantable textiles allow the implanted textile to facilitate differing tissue ingrowth rates at different regions of the implanted graft, resulting in improved medical outcomes and reduced recovery times.

By engineering structural changes in the proximal and distal ends of an endovascular aneurysm repair graft through textile processes, such as, the weave pattern, decreasing density of weft insertion, or increasing porosity, tissue ingrowth can be encouraged in specific areas while maintaining textile characteristics, such as, thickness, and suture strength.

One embodiment provides an implantable woven textile composite formed from a single woven construction. The textile may contain both non-resorbable (i.e. permanent) and/or resorbable yarns in the warp and/or weft. The ratio of non-resorbable yarns to resorbable yarn may be constant or may vary within the textile. The number of ends per inch, picks per inch, or both is varied within the textile resulting in variations in density throughout the textile. The density variations result in variable porosity across the textile structure. The textile composite additionally includes a resorbable coating, which fills the pores and results in a water impermeable barrier.

Another embodiment provides an implantable woven textile formed from a plurality of woven constructions. The textile may contain both non-resorbable (i.e. permanent) and/or resorbable yarns in the warp and/or weft. The ratio of non-resorbable yarns to resorbable yarn may be constant or may vary within the textile. The number of ends per inch, picks per inch, or both is optionally varied within the textile resulting in variations in density throughout the textile. The differing weaves and optional variations of ends and picks provide density variations resulting in variable porosity across the textile structure. In some embodiments, the textile is a composite textile that additionally includes a resorbable coating overlying and in intimate contact with the textile, the coating filling the pores to provide a water impermeable barrier.

The textile may be formed from various woven constructions including a plain weave, twill weave, rib weave (e.g., warp rib or weft rib), satin weave, leno weave, mock leno weave, crepe weave, basket weave and/or herringbone weave. In some embodiments, the textile is formed from a plain weave, a 2×2 twill weave, weft rib, or satin weave. It will be appreciated that in some embodiments the textile may have a uniform weave construction substantially throughout, while in other embodiments the textile may be a continuous weave that comprises two or more different weave constructions at different regions of the textile that are connected by one or more transition regions.

The yarn can be monofilament or multi-filament and formed of any material of construction that can be suitably used for implantable textiles, including, for example, poly(ethylene terephthalate) (PET), polytetrafluoroethylene (PTFE), and collagen, as well as a variety of resorbable materials including PGA, PGS, PLA and PCL, for example that may be interwoven with more permanent materials such as PET and PTFE. In certain presently preferred embodiments, the yarn includes PET. In some embodiments, the yarn has a substantially round cross-section.

The denier of the yarn may range between 10 and 200 denier. In some embodiments, the denier of the yarn is between 10 and 50 denier, such as at least 10 denier, at least 12 denier, at least 14 denier, at least 15 denier, at least 16 denier, at least 17 denier, at least 18 denier, at least 19 denier, at least 20 denier, at least 21 denier, at least 22 denier, at least 24 denier, about 30 denier, about 40 denier, less than 50 denier, and any range or subrange of any of the foregoing.

Textiles in accordance with exemplary embodiments have an end count preferably less than 700 ends per inch (EPI) and with less than 400 picks per inch (PPI). In some embodiments, the textile is a woven textile having less than 600 EPI, less than 500 EPI, less than 400 EPI, between 120-320 EPI and less than 300 PPI, less than 200 PPI, less than 150 PPI, between 80-148 PPI on loom or any range or sub-range of any of the foregoing. It will be appreciated that while the EPI and PPI generally fall within the foregoing ranges, the specific number of EPI and PPI may vary between different regions within the textile as described more fully herein with respect to certain embodiments. Optionally, several warp ends may be bundled and woven as one to reinforce each end. This may result in textile structures having improved suture retention strength characteristics.

After weaving, the resulting textile may be scoured to remove any lubrications or stains on the fabric and then heat set, such as, for example, on a stainless-steel mandrel to shape set the textile into a tubular construct. In one embodiment, the fabric is cleaned and then heat set at about 205° C. (about 400° F.) for shape setting and maintaining dimensional stability.

The resulting bare textile has a thickness that generally ranges between 35 and 300 micrometers, and in some embodiments is between 110 and 280 micrometers.

It will be appreciated that in various medical applications, such as heart valve replacement or repair, it may be desirable to have a water impermeable barrier. Thus, the bare textile may be coated after weaving with a bioresorbable or non-bioresorbable materials to reduce water permeability, thereby forming a composite textile.

Suitable coating materials include various elastomers, regenerative materials, tissue ingrowth-promoting materials, friction-reducing materials, and friction-enhancing materials. Suitable non-bioresorbable coating materials include, but are not limited to, polyurethanes (PU), silicones, and ethylene vinyl acetate (EVA). Suitable synthetically-derived resorbable materials include, but are not limited to, polycaprolactone (PCL), polylactic acid (PLA), polyglycolic acid (PGA), poly(lactic-co-glycolic acid) (PLGA), poly(glycerol sebacate) (PGS), lysine-poly(glycerol sebacate) (KPGS), poly(glycerol sebacate urethane) (PGSU), amino-acid incorporated PGS, and combinations thereof. Suitable naturally-derived resorbable materials include, but are not limited to, collagen, fibrin, elastin, hyaluronic acid, glycosaminoglycans, proteoglycans, polysaccharides, proteins, amino acids, extracellular matrix components, and combinations thereof. In some embodiments, the coating may be applied by spray coating, dip coating, or lamination techniques.

In some embodiments, the water permeability of the textile is less than 500 mL/min/cm², less than 400 mL/min/cm², less than 375 mL/min/cm², less than 350 mL/min/cm², less than 325 mL/min/cm², less than 300 mL/min/cm², less than 275 mL/min/cm², less than 250 mL/min/cm², less than 225 mL/min/cm², less than 200 mL/min/cm², less than 150 mL/min/cm², less than 100 mL/min/cm², less than 75 mL/min/cm², less than 50 mL/min/cm², less than 30 mL/min/cm², less than 20 mL/min/cm², less than 10 mL/min/cm², less than 5 mL/min/cm², less than 3 mL/min/cm², and/or less than 1 mL/min/cm².

The incorporation of a fused coating onto the textile can temporarily seal the pores of the textile to provide a low water permeability textile. When implanted into the body, the coated textile provides a temporary liquid impermeable barrier at the surgical site until the ingrowth of the body's natural cells. By selecting one or more biomaterials with a polymer degradation rate that matches the regeneration rate of one or more nearby native tissues, the composite can be tailored with zones that encourage ingrowth of various tissues selectively, in a spatiotemporal manner. This degradation rate may be as short as one week, such as a natural material like fibrin, and may be as long as one to two years, such as a synthetic material like polycaprolactone (PCL). PGS is of particular utility since its degradation rate can be tuned by changing the glycerol:sebacic acid stoichiometry.

Besides the degradation rate of the fused biomaterial, the mechanical properties of the biomaterial also affect tissue ingrowth and regeneration. Similarly, to the degradation rate, various cell types have a preference for the stiffness of substrate they will grow onto and into. Thus, biomaterial selection can be tailored to encourage ingrowth of various tissues selectively. Substrate stiffness may also influence stem cell differentiation, so multipotent or precursor cells in native tissues can be driven down desired differentiation pathways once they encounter the substrate. Again, PGS may be preferred in this application because its mechanical properties can be tuned by altering the glycerol:sebacic acid stoichiometry.

Additionally, the textile pore dimensions, pore shape, and fiber texture all influence cell infiltration. Various cell types have a preference for certain pore sizes, shapes, aspect ratios, and/or alignments. This allows textile design parameters to be selected that preferentially, in a spatiotemporal manner, induce and encourage ingrowth of specific cell types, selectively drive differentiation of precursor cells into mature tissue, and mimic the endogenous tissue environment during regeneration.

In exemplary embodiments, native tissue grows within the structure in an effort for the grafts to properly seal to the surrounding native tissue to avoid graft migration and leakage post-surgery. Cell infiltration cannot occur in pores that are too small, such as 5 micrometers or below. Pores that are too large, such as 1 millimeter and greater, can experience cell infiltration, but the pore interior is too large for excreted extracellular matrix (ECM) to infill completely and so the tissue-graft interface is leaky and poorly adhered. Cell types are known to migrate in, remain viable, proliferate, differentiate, excrete ECM, and generate mature tissue most optimally in pores of specific size ranges and shapes. Similar cell behavior occurs with textures, patterns, and alignments of pore, fibers, and surfaces. For example, bone regeneration by osteoblasts occurs best with a pore size between 150-350 micrometers, while cartilage regeneration by chondrocytes occurs best with a pore size between 50-250 micrometers. Beyond the cellular level, when considering graft anchoring at the tissue level, fixation between orthopedic implants and surrounding bone tissue is best achieved under the shortest duration when the implant is designed with the proper porosity, pore size, and surface roughness for bone tissue ingrowth, such as 600 micrometer pore size and 65% porosity. For peripheral axons, regeneration occurs best in long pores between 200-750 micrometers, whereas axon ingrowth and outgrowth only require 20-70 micrometer pores. Vessel regeneration by endothelial cells and pericytes occurs best with guided aligning structural cues, small pore sizes below 60 micrometers, and small interpore distances below 50 micrometer. A higher ratio of pore size to endothelial cell size leads to more successful cell migration and invasion. Cardiovascular smooth muscle cells are cultivated best on scaffolds with a pore size between 40-150 micrometers. Thus, exemplary embodiments can be used to provide engineered textiles with different characteristics, such as, for example, varying porosity within the fabric and creating open spaces which native cells can infiltrate.

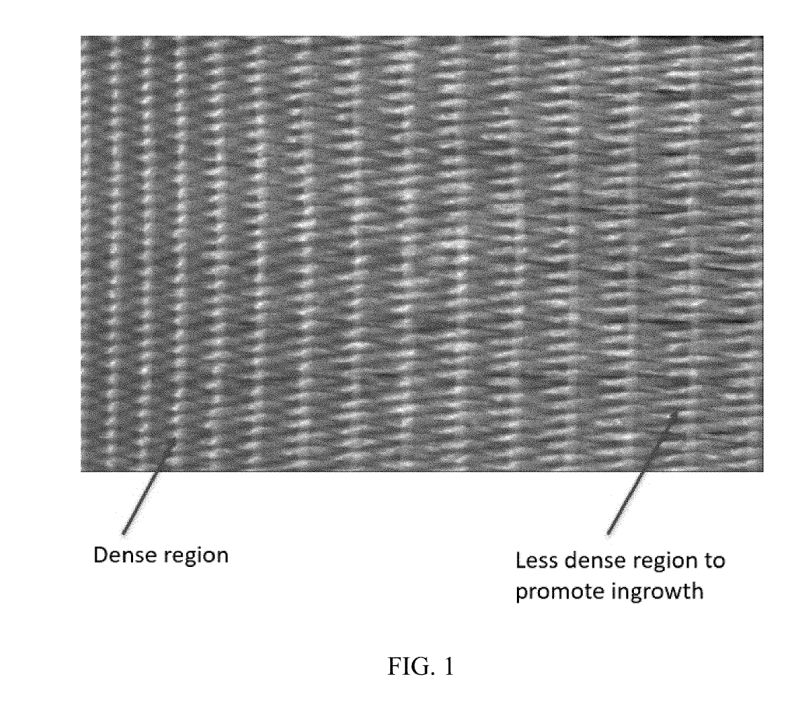

As shown in FIG. 1, varying the density of the fabric construct can decrease or increase the amount of material in said textile, which in turn provides the ability to open the porosity in certain regions of the textile to allow larger spaces between yarns in which tissue can grow.

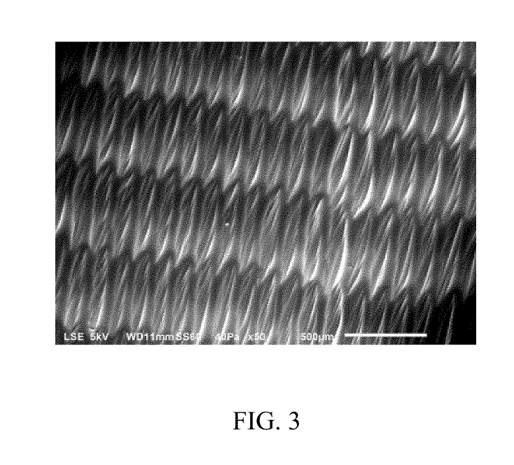

In one embodiment, density changes by a gradual decrease in pick insertion (i.e. an ombre technique), thereby gradually increasing the size of the pores. The textile is subsequently coated with a regenerative material, preferably PGS, to create a water impermeable composite, as shown in FIG. 3. Porosity can be regulated over time by introducing yarns of resorbable biomaterials, such as, for example polyglycolic acid (PGA), or regenerative materials, such as PGS yarn, in combination with permanent materials, such as polyethylene terephthalate (PET), in designated areas. In embodiments using both a resorbable material and a non-absorbable material, the resorbable material is resorbed by the body in time, which creates an opening where that material once was. In some embodiments, use of regenerative yarns in specific regions may encourage the body to not only resorb material but also grow native tissue by encouraging endotheliosis.

In one embodiment, a plurality of resorbable materials may be used in combination with non-resorbable materials, such as weaving yarns of a first resorbable material in a pre-determined manner interspersed between non-resorbable yarns during textile formation followed by coating the bare textile with a second resorbable material in forming the composite textile. For example, after implantation, a resorbable coating material, such as a PGS coating, may be resorbed by the body within a few weeks, allowing ingrowth over a large area of the implant during the initial recovery after surgery while a yarn of a different resorbable material that is more slowly resorbed, such as PGA, may provide regions of slowly increasing porosity within the textile, allowing continued tissue ingrowth for one to two years or more. The remaining non-resorbable materials ultimately provide a permanent scaffold for tissue growth.

In exemplary embodiments, not only is porosity varied within designated areas of the graft, bioresorbable material is also introduced within the proximal and distal regions of the graft. The entire graft may be further coated with a regenerative material in order to minimize inflammation. The regenerative coating also allows this composite to have a temporarily water-impermeable feature. For example, the composite may remain water-impermeable for four to six weeks, or longer or shorter as needed to allow enough time for tissue regeneration. The composite acts as a scaffold, providing a structure for cells to grow on and through the graft in the designated bioresorbable areas. The ongoing increase in porosity may also enhance the flow of nutrients and growth factors to the affected regions.

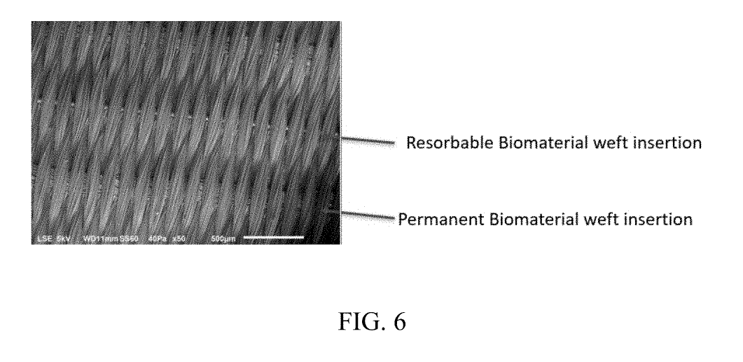

In an embodiment, the composite graft uses the technique of varied density within the same weave structure, as well as introducing resorbable and or regenerative fibers into the areas of larger porosity to add another level of cell growth integration into the construct, as shown in FIG. 6. As the biomaterials within the stent graft start to degrade, native cells integrate through the graft and adhere to the tissue wall, resulting in less chance of sealing failures and shifting within the vasculature.

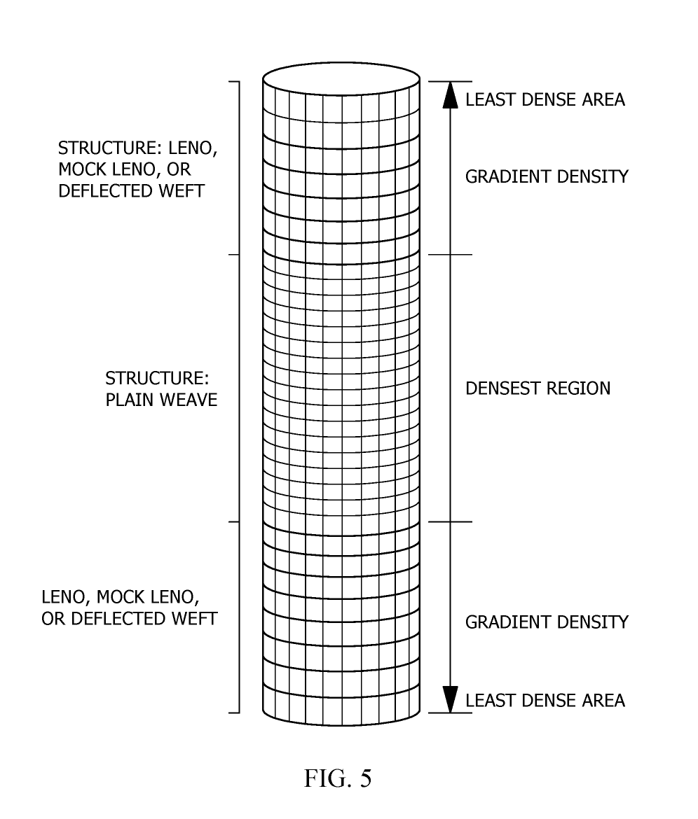

FIG. 5 describes an embodiment of a woven tubular composite graft having a central region of a first construction. In the example of FIG. 5, the first construction is a plain weave construction. The woven graft includes a distal end region formed by a second construction. In the example of FIG. 5 the distal end region may be a leno, mock leno, or deflected weft construction. The yarn density varies within the distal end region. In the example of FIG. 5 the yarn density decreases in a direction away from the central region. Similarly, to the distal region, a proximal end region is formed by a third construction. In the example of FIG. 5 the proximal end region may be a leno, mock leno, or deflected weft construction. The yarn density varies within the proximal end region. In the example of FIG. 5 the yarn density decreases in a direction away from the central region. The second and third constructions may be the same or different.

Resorbable yarns may be inserted into the warp or weft at varying or constant densities of the tubular textile of FIG. 5. For example, at the boundary of the central and distal regions a resorbable yarns may be inserted into the weft, replacing a non-resorbable yarn once per ten picks.

The rate of insertion may be varied over the region to, for example, result in a non-resorbable yarn replacing a non-resorbable yarn once per three picks at an edge of the textile. The rate of insertion may be held constant, linearly increased or decreased, or non-linearly increased or decreased over the length of the region. In some embodiments, the rate of insertion is increased nearer the distal and proximal edges.

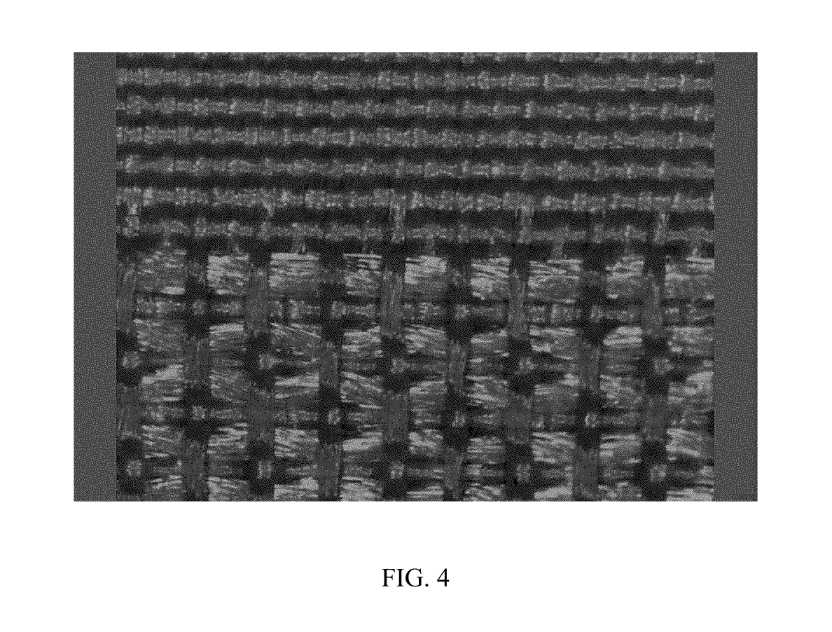

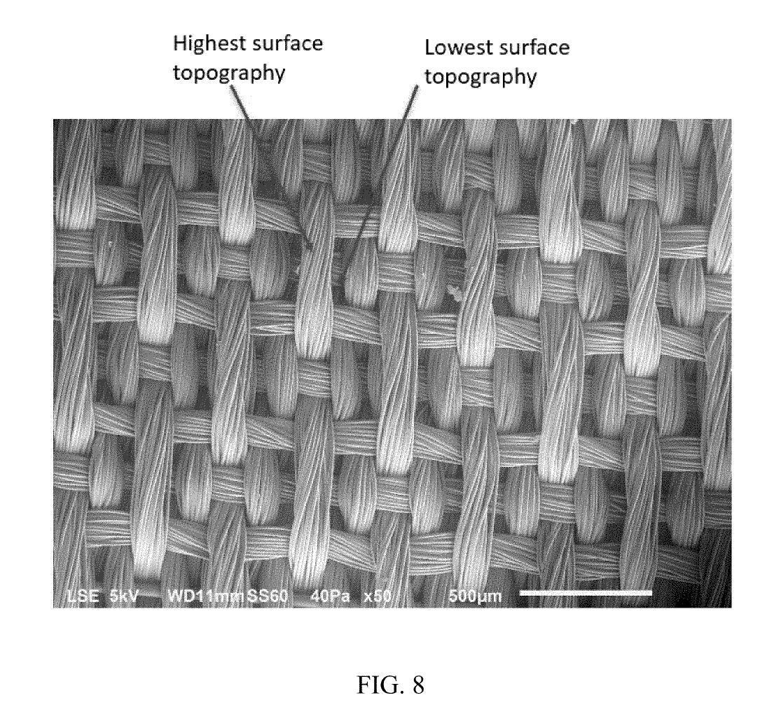

Another embodiment for encouraging targeted cellular growth is by altering the topography of the surface by using at least two different weave structures, as shown in FIG. 4. In one embodiment, the center of the graft may exhibit a flatter topography, such as a plain weave, where there are not as many hills and valleys created in a single layer woven structure. Conversely, the targeted areas for ingrowth may use a variable/fluctuating surface with pockets that would fill with the regenerative material once coated. One technique includes the formation of a deflected weft weave structure, as shown in FIG. 8. After coating, the valleys of this weave provide for pools of regenerative material in specific areas to form during coating. These pools may in turn provide a locus to encourage cellular growth and attachment of the graft to the tissue wall.

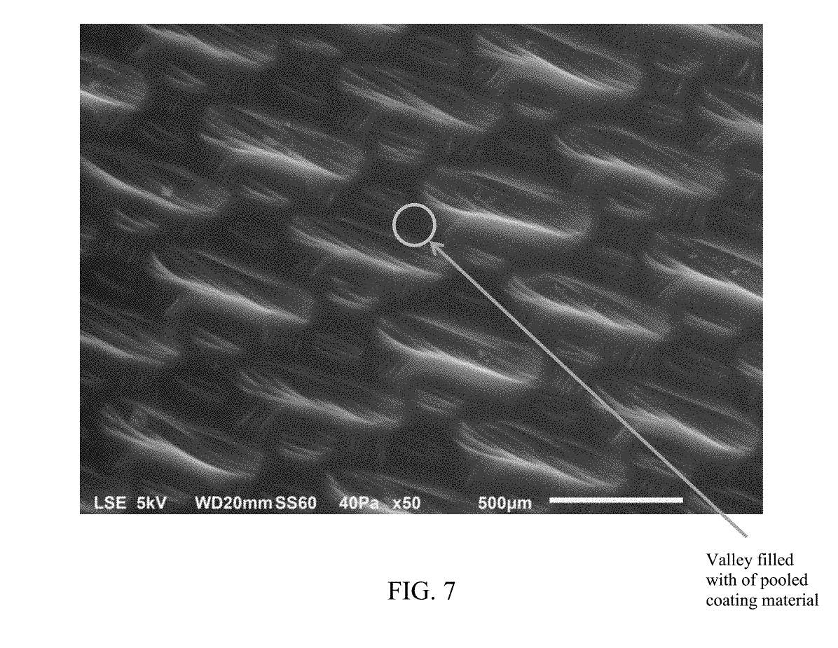

The target size of the pools can match the size of the tissue functional unit. The functional unit is defined as the smallest scale a tissue can be while still retaining its function and properties. Alternatively, the target size of the pools match what is optimal for ingrowth of a particular cell type. Alternatively, the target size of the pools are designed to contain and/or release a defined amount of nutrients, anti-oxidants, oxygen, chemotactic agents, growth factors, any bioactive small or large molecules, and/or any inactive ingredients, as shown in FIG. 7.

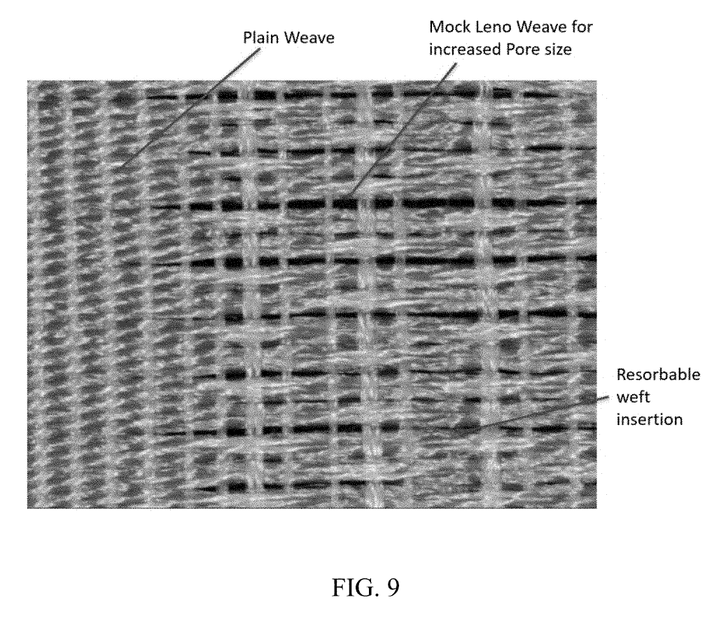

In an embodiment, a composite graft may be formed by combining at least two weave structures and introducing resorbable biomaterials into the proximal and distal regions by weft insertion. In some embodiments, the weft insertion is non-uniform across a region, to encourage cellular growth in specific areas, as shown in FIG. 6. Resorbable materials may be inserted at varying frequencies throughout the textile to promote ingrowth. Alternatively, or additionally, the number of ends per inch and/or the number of picks per inch may be varied to alter the density of various regions of the graft in the proximal and distal regions to increase pore size, which may be accomplished by having regions of different weave construction, as shown in FIG. 9.

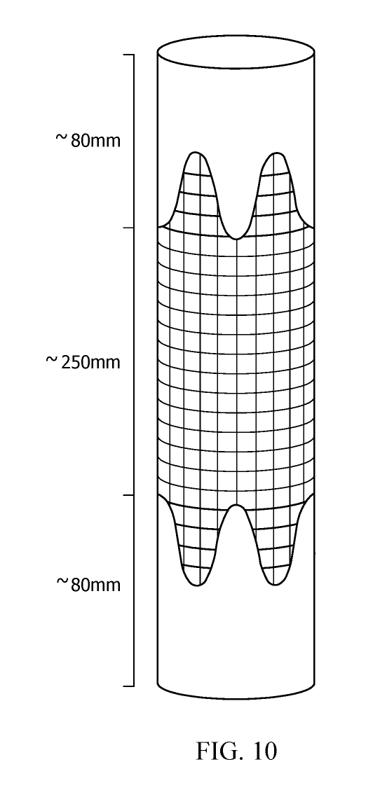

Another embodiment may use linear shaping between the two differing weave structure profiles, which may create a stronger bond between the body of the graft and surface ingrowth. Linear shaping is shown in FIG. 10. FIG. 10 is an example of a woven tubular composite implantable textile. In the example of FIG. 10 the textile includes a central region of a first construction. In the example of FIG. 10, the first construction is a double cloth plain weave construction formed from 20 denier warp yarns and 20 denier weft yarns. The central region has a length of about 250 millimeters. The textile also includes distal and proximal end regions having a construction of an alternating plain weave and warp faced twill weave. The construction is formed from formed from 20 denier warp yarns and 20 denier weft yarns. The weave variation increases the surface profile of the textile and may promote increase tissue ingrowth after implantation. The distal and proximal end regions are each about 80 millimeters in length. The boundary between the central region and the distal and proximal edge regions may additionally possess linear shaping to increase the bond strength between the body of the graft and the tissue ingrowth.

In exemplary embodiments, when coating a low profile textile structure (e.g., a bare textile having a thickness of about 60 microns or less), an elastomeric polymer may be used that elongates with the textile and maintains its low water permeability characteristic. The elastomeric coating maintains its integrity while conforming to unique geometries in response to the body's internal movements and pulsation.

In some embodiments, elastomeric fibers may also be integrated into the woven textile to enhance pulsatile behavior. In some embodiments, the elastomeric fibers may include Polyurethane (PU), vicryl (polygalactin 910), or polycaprolactone (PCL). The elastomeric fibers may be integrated into the warp or weft of the textile.

In some embodiments, the coating may be composed of a cell proliferation-promoting material, such as PGS/nutrient/amino acid/citric acid adduct polymers that are formulated to enhance cell proliferation, viability, differentiation, healing, and combinations thereof.

In an embodiment, a woven graft is formed as a woven tubular fabric designed to be implanted into a mammalian body. In particular, the graft is designed to be implanted into a human patient. In an embodiment, the graft is a continuously woven tubular element.

As discussed further below, the graft is formed so that the fabric has a gradient of yarn densities along at least a portion of the length of the graft. In one embodiment, the ends per inch of the warp yarns in the graft is varied along the length of the graft. In this way, features such as the porosity and flexibility of the graft, may be customized along the length of the graft to promote tissue in-growth. The graft may be formed from any of a number of natural or synthetic fibers.

More than one type of yarn may be used as warp yarns or weft yarns. The weft yarns may differ from the warp yarns. Additionally, more than one type of weft yarn may be used. In some embodiments, the warp and weft yarns include combinations of regenerative and non-regenerative yarns. In one embodiment, the warp and weft yarns include polyethylene terephthalate (PET) yarn in which poly(glycerol sebacate) (PGS) yarn is interspersed in a pre-determined manner. The yarns may be monofilament or multi-filament. In one embodiment, the polyester fibers include 1/40/27/12Z PET multi-filament fibers.

In an embodiment, the graft is a seamless lumen formed as a flat woven tubular textile. The weave may be any of a variety of weaves, including, but not limited to plain, basket and twill weaves. In some embodiments, the graft is formed of a plain double cloth weave forming a flattened tubular structure. The characteristics of the weave pattern may vary depending upon the application for the graft. However, in one embodiment, the graft is formed so that the walls are substantially impermeable to fluid, so that the graft forms a lumen that is substantially fluid-tight along its length with an inlet and an outlet. For example, when used in a vascular application, the walls of the graft are substantially impermeable to blood so that the graft forms a conduit permitting the flow of blood along the axis of the graft while impeding blood leakage through the sidewalls of the graft.

To provide a fluid-tight textile, the fabric comprises approximately 150-350 ends per inch (“EPI”) at approximately 100-200 picks per inch (“PPI”) for each face. Since the flat woven tube comprises two faces, the total end count for the graft is approximately 300-700 ends at approximately 200-400 PPI. More specifically, the fabric may comprise approximately 200-300 EPI at 125-175 PPI for each face. In the present instance, the fabric comprises approximately 225-275 EPI at approximately 150 PPI for each face.

In some embodiments, the graft is woven on a loom configured to produce a plain weave double cloth textile. The loom may be any of a variety of types, including, but not limited to a jacquard loom, a circular loom or a dobby loom. In one embodiment, the graft is produced on a dobby loom. The loom includes a plurality of harnesses for controlling a plurality of heddles that control the warp yarns.

When using a dobby loom, each harness controls a plurality of heddles b etween a first position and a second position, such as a raised position and a lowered position. The number of harnesses may vary depending on the size and configuration of the graft. In the present instance, the loom utilizes twenty harnesses.

From the harnesses, the warp yarns pass through a reed having a plurality of slots or dents. The reed may be a straight reed or a tapered reed. In one embodiment, the reed is a tapered reed so that the reed tapers from a first width down to a second width that is narrower than the first width. Specifically, the reed is widest at the upper end of the reed and is narrowest at the lower end of the reed. In particular, the dents of the straight reed are spaced out across the width of the reed so that each dent is substantially the same width. The dents of a tapered reed taper from the top of the reed to the bottom of the reed so that the dents are widest at the top of the reed and narrowest at the bottom. Alternatively, the reed may be inverted so that the dents and the reed are widest at the bottom and narrowest at the top.

The position of the reed is controlled by a controller that is operable to selectively move the reed up or down to vary the number of ends per inch of the woven fabric. Specifically, moving the reed upwardly pulls or squeezes the warp yarns inwardly, increasing the ends per inch of the fabric if the number of warp yarns remains constant. Similarly, moving the reed downwardly pulls the warp yarns outwardly decreasing the number of ends per inch of the fabric if the number of warp yarns remains constant. Alternatively, when using a straight reed, the number of warp ends per inch may be varied as the number of warp yarns is increased and/or decreased along the length of the fabric. The controller may control the timing and rate of reed movement depending on a number of variables, including, but not limited to: the configuration of the graft, the desired density and the number and timing of dropped warp yarns.

The loom also comprises one or more shuttles for weaving the weft yarns onto the warp yarns. When a single lumen graft is formed, a single shuttle may be used. When a multiple lumen graft is formed, multiple shuttles may be used as discussed further below.

Each pass of the shuttle across the warp yarns comprises a pick. When weaving a double cloth textile to form a tubular structure, a pass of the shuttle back and forth completes two pick lines which form a single continuous thread circumscribing the circumference of the tubular fabric. As the shuttle moves forward, it weaves a pick line on the front face of the fabric. As the shuttle returns, it weaves a pick line on the rear face of the fabric. By raising and lowering the warp yarns after each forward pass and return pass of the shuttle, the weft yarn from the shuttle continuously weaves from the front face to the rear face without a break or seam.

After the shuttle weaves the weft yarn, the loom moves the reed toward the fell to beat the fabric. The leading edge of the woven fabric is attached to a take-up roll so that the fabric is continuously wound onto the take-up roll as the fabric is finished. The take-up roll also maintains tension on the warp yarns so that the warp yarns are under appropriate tension to weave the fabric. For instance, the take-up roll may be rotated regularly as the weaving process continues. As the take-up roll rotates, the woven material is wound onto the take-up roll, thereby applying tension to the warp yarns.

As discussed above, to weave the fabric, a controller controls the operation of the harnesses, the reed and the shuttle(s) to weave the fabric that forms the graft.

For instance, in order to form a double cloth weave of varying density, the pattern for dropping the yarns from the weave may vary depending on the desired density along the length of the woven fabric. In particular, the position of the dropped yarns may be controlled so that the dropped yarns are all removed from the weave at substantially the same time. Alternatively, the dropped yarns may be removed gradually.

For example, the dropped yarns may be removed in groups. The total number of dropped yarns may be divided into two or more groups of dropped yarns. At a certain point along the length of the woven fabric, a first group of dropped yarns may be controlled so that the first group of dropped yarns is not woven with the weft yarn.

After all of the dropped yarns are dropped from the weaving pattern, the weaving may continue weaving the base yarns. The result is a flat woven tubular textile having at least two sections: a first section in which the base and dropped yarns are woven in the fabric; and a second section in which the base yarns are woven without the dropped yarns. In this way, the dropped yarns are interlaced with the weft yarns in the first section, but, are outside of the weave pattern in the second section.

The dropped yarns may be added back into the weave in a similar manner to how the yarns are dropped from the weave as described above. In other words, the process for adding the dropped yarns back into the weave may proceed by reversing the process used to drop the dropped yarns. Additionally, while the dropped yarns are added back into the weave, the controller may control the reed to increase the width of the dents through which the warp yarns are drawn, if desired. In one embodiment, the dropped yarns and the reed may be controlled so that a graft is woven in reverse of the process described above. In this way, the weaving process forms a graft having a density gradient from low to high and then from high to low. This process repeats for the length of the warp yarns to produce a series grafts in which the weaving alternates between weaving the densest portion first and dropping yarns to weaving the least dense portion first and adding yarns.

After the yarns are added back in so that the fabric is as dense as the first section, the weave may not be optimally uniform to provide the uniformly smooth wall surface that is desired for the graft. However, by continuing to weave the fabric at a generally uniform density after the yarns are added back in, the weaving process generally settles into a uniformly woven fabric that provides the uniformly smooth wall surface desired for the graft. Accordingly, between the end of a first graft and the beginning of the next graft, the transition section extends further than the length of the woven section in which the yarns are added back into the web.

As previously described, porosity is manipulated in exemplary embodiments by changing a weave structure, by removing ends and/or removing picks, and/or by introducing resorbable biomaterials, such as, for example polyglycolic acid (PGA), or regenerative materials, such as, for example, poly(glycerol sebacate) (PGS) yarn, in combination with permanent materials, such as, for example, polyethylene terephthalate (PET), in designated areas.

In exemplary embodiments, not only is porosity varied within designated areas of the graft, bioresorbable material is also introduced within the graft. In exemplary embodiments, the entire graft is coated with a bioresorbable material in order to minimize inflammation and encourage the timing of tissue regeneration in specific areas, while creating a water-impermeable ultra-low profile composite.

The textiles produced by the materials and techniques described herein are described primarily for use in endovascular applications, such as straight or bifurcated woven implantable grafts; endovascular aneurysm repair (EVAR) and transcatheter aortic valve replacement (TAVR) procedures, but may also be used in various other applications including hernia repair, urology, incontinence, and breast augmentation; coated braided sutures and tethers, all by way of example.

While the invention has been described with reference to one or more embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

1. A woven implantable textile comprising:
a continuous length of woven textile having a first region and a second region of a common face;
the first woven region having a first ends per inch in a warp and a first picks per inch in a weft, defining a first density;
the second woven region having a second ends per inch in the warp and a second picks per inch in the weft, defining a second density different from the first density;
a bioresorbable coating overlying the first and second woven regions to form a composite textile,
wherein the weave construction of the first woven region and the second woven region are the same.

2. The textile of claim 1:
wherein the first ends per inch and the second ends per inch are the same; and
wherein the first picks per inch and the second picks per inch are different.

3. The textile of claim 1, further comprising a gradient of picks in a transition region between the first woven region and the second woven region.

4. The textile of claim 1, wherein the textile is less than 100 micrometers thick.

5. The textile of claim 1, wherein the weave construction is a plain weave, a twill weave, a warp rib weave, a weft rib weave, a leno weave, deflected weft weave, or a mock leno weave.

6. The textile of claim 5, wherein the weave construction is a weft rib weave.

7. The textile of claim 1, wherein the textile includes a polyethylene terephthalate yarn in the warp.

8. The textile of claim 1, wherein the polyethylene terephthalate yarn denier is between 15 denier and 50 denier.

9. The textile of claim 1, wherein the textile includes a resorbable yarn in the weft.

10. The textile of claim 1, wherein the textile exhibits a water permeability of less than 500 milliliters/minute/centimeter².

11. The textile of claim 1, wherein the textile exhibits a water permeability of less than 5 milliliters/minute/centimeter².

12. The textile of claim 1, wherein the textile exhibits an average pore size of 10 micrometers to 800 micrometers in at least one region.

13. The textile of claim 1, wherein the bioresorbable coating is selected from the group consisting of polycaprolactone (PCL), polylactic acid (PLA), polyglycolic acid (PGA), poly(lactic-co-glycolic acid) (PLGA), poly(glycerol sebacate) (PGS), Lysine-poly(glycerol sebacate) (KPGS), poly(glycerol sebacate urethane) (PGSU), amino-acid incorporated PGS, and combinations thereof.

14. A woven implantable textile comprising:
a continuous length of woven textile having a first region and a second region of a common face;
the first woven region formed from a first woven construction;
the second woven region formed from a second woven construction;
wherein the weave construction of the first woven region is different from the weave construction of the second woven region.

15. The textile of claim 14:
wherein the first woven construction is selected from the group consisting of a plain weave, a twill weave, a warp rib weave, a weft rib weave, a leno weave, and a mock leno weave; and
wherein the second woven construction is selected from the group consisting of a plain weave, a twill weave, a warp rib weave, a weft rib weave, a leno weave, a deflected weft weave and a mock leno weave.

16. The textile of claim 15, wherein the first woven construction is a plain weave and the second woven construction is a weft rib weave.

17. The textile of claim 14, further comprising a bioresorbable coating overlying the textile, the coating selected from the group consisting of polycaprolactone (PCL), polylactic acid (PLA), polyglycolic acid (PGA), poly(lactic-co-glycolic acid) (PLGA), poly(glycerol sebacate) (PGS), Lysine-poly(glycerol sebacate) (KPGS), poly(glycerol sebacate urethane) (PGSU), amino-acid incorporated PGS, and combinations thereof, thereby forming a composite textile.

18. The textile of claim 17, wherein the composite textile exhibits a water permeability of less than 5 milliliters/minute/centimeter².

19. The textile of claim 14, further comprising a coating overlying the textile to form a composite textile, the composite textile being less than 100 micrometers thick and having a water permeability of less than 5 milliliters/minute/centimeter².

20. The textile of claim 14, wherein the textile exhibits an average pore size of 10 micrometers to 800 micrometers in at least one woven region.

21. The textile of claim 14, wherein the textile includes a polyethylene terephthalate yarn in the warp.

22. The textile of claim 14, wherein the polyethylene terephthalate yarn denier is between 15 denier and 50 denier.

23. The textile of claim 14, wherein the textile includes a resorbable yarn in the weft.
